(19) United States
(12) Patent Application Publication
(10) Pub. No.: US 2025/0281061 A1
(43) Pub. Date: Sep. 11, 2025

(54) MONITORING DEVICE AND METHOD FOR OPERATING A MONITORING DEVICE

(71) Applicant: Bearcover GmbH, Berlin (DE)

(72) Inventors: Daniel Flynn, Berlin (DE); Ben Duffy, Berlin (DE); Fernando German Torales Chorne, Berlin (DE)

(21) Appl. No.: 18/859,975

(22) PCT Filed: Apr. 21, 2023
(86) PCT No.: PCT/EP2023/060417

§ 371 Date: Oct. 24, 2024

(30) Foreign Application Priority Data
Apr. 27, 2022 (DE) 10 2022 110 175.4

(51) Int. Cl. A61B 5/05; A61B 5/11; A61B 5/113; G01C 21/20; G01S 13/02; G01S 13/58

(52) U.S. Cl. CPC A61B 5/05; A61B 5/1123; A61B 5/113; G01C 21/206; G01S 13/0209; G01S 13/582

(57) ABSTRACT

In an embodiment a monitoring device includes an autonomous robot unit configured to navigate the monitoring device along a navigation path, a radar sensor unit configured to detect a person using radar technology and at least one processing unit configured to detect an anomalous situation based on a detection result of the radar sensor unit and to generate a monitoring signal in response to detecting the anomalous situation.

### CROSS-REFERENCE TO RELATED APPLICATIONS

This patent application is a national phase filing under section 371 of PCT/EP2023/060417, filed Apr. 21, 2023, which claims the priority of German patent application 102022110175.4, filed Apr. 27, 2022, each of which is incorporated herein by reference in its entirety.

### TECHNICAL FIELD

The present disclosure relates to a monitoring device and a method for operating such monitoring device, in the field of monitoring a person.

### BACKGROUND

There are many situations, in which monitoring a person may be required. For example in nursing homes or hospital, caregivers are required to ensure safety and wellbeing of their residents and patients. Depending on a health situation of such residents and patients, for example, it may be required that supervision is provided around the clock, at least occasionally.

Since providing such supervision with healthcare workers checking the rooms of such patients and residents requires a large workload for healthcare workers and reduces the time and effort that can be provided to personal needs of other patients or residents, monitoring systems to supervise residents in nursing homes and patients in hospitals were developed. Those systems comprise, for example, cameras installed in rooms of patients and residents, or wearable devices on the patients or residents directly. Both solutions, however, come with a large financial expense and can significantly violate personal privacy of the residents and patients.

### SUMMARY

Embodiments provide a monitoring device and an operating method for a monitoring device with which a person may be monitored while reducing the above-mentioned problems.

According to a first embodiment, a monitoring device comprises an autonomous robot unit, arranged to navigate the monitoring device along a navigation path. The monitoring device further comprises a radar sensor unit, arranged to detect a person using radar technology. The monitoring device further comprises at least one processing unit, arranged to detect an anomalous situation based on a detection result of the radar sensor unit, and to generate a monitoring signal in case an anomalous situation has been detected.

An advantage of the monitoring device described herein is that by autonomously navigating the device and using radar technology to detect a person, no monitoring systems need to be installed inside a room of a resident or patient or on a person itself. Invasion of personal space of a resident or patient, therefore, is significantly reduced.

Moreover, since one such monitoring device is arranged to navigate autonomously along its navigation path and perform person detection using the radar sensor unit, one such monitoring device may be used to monitor a large number of residents or patients. Therefore, financial expenses in the field of resident and/or patient monitoring may be significantly reduced.

An anomalous situation in the context of this application may be, for example, a situation in which a person should be detected but cannot be detected, i.e. it is assumed that the person is missing. Or that a person is detected where no person should be detected, or a number of persons detected is higher than a number of persons that should be detected.

The navigation path along which the monitoring device is navigated using the autonomous robot unit may be pre-stored in a memory unit of the monitoring device and/or may be calculated by the autonomous robot unit during navigation, based on, for example, stored location map information of an environment in which the monitoring device is navigated. Alternatively, however, other navigation procedures are also possible. Navigation of the monitoring device may be done, in general, according to any navigation technique known in the art of autonomous robot navigation.

According to at least one embodiment, the radar sensor unit comprises at least one ultra-wideband, UWB, radio sensor.

The use of an UWB radio sensor in the radar sensor unit, i.e., using UWB radar technology for detecting a person, may be advantageous because UWB sensors may achieve a high accuracy in detecting a person, in particular in an indoor environment. An UWB sensor may use an UWB signal for radar detection with either a fractional bandwidth of 20% of the center frequency or more than 500 MHz. Further advantages of using an UWB sensor may be: a high range resolution, good penetration characteristics, good immunity against multipath interference, ability to reject clutter, high spatial resolution, and discriminate between targets close to each other.

According to at least one embodiment, the autonomous robot unit comprises at least one of the following:
a light imaging, detection and ranging, LIDAR, sensor,
a depth sensor,
an RGB camera, and
a positioning sensor.

Using any one or a combination of two or more of the above allows the autonomous robot unit to navigate efficiently and safely along the navigation path.

According to at least one embodiment, the radar sensor unit is arranged to perform Doppler radar detection to detect a walking movement of a person and/or a breathing movement of a person.

For Doppler radar detection of movement of a person, dynamic threshold classifiers may be used, e.g. using a constant false alarm rate, CFAR, algorithm. If there is relative motion between the radar sensor unit, i.e. the monitoring device, and a detected target, then the frequency of the electromagnetic wave reflected from the target and received by the radar sensor unit will be different from the frequency of the wave transmitted from the radar (Doppler effect). The Doppler shift is measured by performing a spectral analysis of the received signal in every range increment. Hence, movement of a person may be detected using such Doppler radar detection.

Given that a lot of data may be collected using such monitoring device in, for example a nursing home or hospital, this data may be used with data-driven methods, e.g. Deep Learning, to classify and track certain human behaviour. For example, it may be classified which posture a person is in or which activity they are performing, e.g. sitting, standing, talking, walking, lying. Sleep monitoring and directly detecting falls from learned data are also possible applications that can be achieved with such data-driven methods.

The human physiology provides constraints of frequencies and displacement size of different kinds of movements, which enable the classification of different movement detections as walking movement or breathing movement. A quick movement of a high radar cross-section, RCS, i.e., a part of the human body that reflects the radar waves most directly, may be used to determine a walking movement of a person. Small periodic movement of a chest or abdominal displacement occurring from a breathing motion may be used to determine a breathing movement of a person.

Based on the above distinction between walking movement and breathing movement of a person, further and more detailed detection of an anomalous situation may be determined. For example, it may be determined whether a walking movement is detected at a time and/or location, where no walking person should be detected. If not walking movement is detected, it may further be determined whether a breathing movement is detected at a time and/or location, where a breathing person should be located.

According to at least one embodiment, the radar sensor unit is arranged to determine at least one of the following:
a range at which a movement of a detected person is located;
a displacement range of a movement of a detected person;
a frequency of a movement of a detected person;
an azimuth angle at which a movement of a detected person is located;
an elevation angle at which a movement of a detected person is located.

With the above determined information (at least one or any combination thereof), a more detailed and more accurate information regarding a detected person may be obtained, and hence a decision whether an anomalous situation is detected or not may be based on more profound information.

An accurate elevation angle and range, for example, may be used to determine a height estimation of the movement. The range may be based on “time of flight” measurements, or time between pulse and return of a pulse from the radar sensor unit. The angles may be inferred from phase differences in signal arrival using multiple transmitters. The range at which a movement of a detected person is located may be measured, for example, with respect to so called range bins.

For example, it may be determined where in a supervised environment a person is exactly located. Hence, based on a scan of the environment and/or stored environmental information on the monitoring device, such as where in a room, for example, a bed is located, it can be determined whether a person is detected in an area of the bed or away from the bed. Based on a time of day, for example, this information may be used to determine whether a situation is an anomalous situation or not.

Moreover, for example based on a determination of a height of a movement above ground, it may be determined whether a person is standing or lying e.g. on a floor, in particular also in combination with a determination whether a walking movement or a breathing movement is detected.

The detected displacement range and/or frequency of a movement of a detected person may be used to distinguish between a walking movement and a breathing movement. For example, “breathing-like” and/or “walking-like” signals may be detected based on an expected frequency and displacement range for a normal breathing/walking movement. Certain thresholds for frequency and displacement range may be defined to determine whether a detected movement is considered as a breathing movement, or a walking movement. A breathing rate may be measured in inhale-exhale cycles per minute.

Further, also the range of a movement of a detected person may be used to distinguish between a walking movement and a breathing movement, since, as mentioned above, each type of movement underlies specific constraints with regard to a range and a velocity of each type of movement.

According to at least one embodiment, the monitoring device further comprises a modem, via which the monitoring device is arranged to send the monitoring signal to a monitoring system.

This way, a monitoring system, for example comprising an application running on a computer, tablet, or smartphone, etc., of a caregiver, may be notified in case an anomalous situation is detected. Alternatively or additionally, the monitoring signal may also be communicated to a caregiver on the monitoring device directly, e.g. via a visual and/or audio alarm, and/or by displaying a message on a display, etc.

According to at least one embodiment, the autonomous robot unit is arranged to navigate the monitoring device along the navigation path in an indoor environment, and the radar sensor unit is arranged to detect a person behind a wall and/or a door.

The above embodiment is in particular advantageous since in nursing homes and hospitals, residents and patients are usually located in indoor environments with closed rooms.

Therefore, the monitoring device can perform safety checks on residents and patients from outside rooms, without the need of doors being opened, etc.

According to a second embodiment, a method for operating a monitoring device comprising an autonomous robot unit, a radar sensor unit and at least one processing unit, comprises the following steps:
navigating, by the autonomous robot unit, along a navigation path;
performing, by the radar sensor unit, at least one radar scan to detect a person using radar technology;
determining, by the processing unit, whether an anomalous situation is detected based on a detection result of the radar sensor unit; and
generating, by the processing unit, a monitoring signal in case an anomalous situation has been detected.

According to at least one embodiment, the performing, by the radar sensor unit, at least one radar scan to detect a person comprises:
performing, by the radar sensor unit, a first scan to detect a walking movement of a person; and
performing, by the radar sensor unit, a second scan to detect a breathing movement of a person.

First and second scan may be performed using different radar parameters to determine whether a respective movement is detected within respective minimum and maximum thresholds for frequency and/or displacement range. Additionally, for multiple scans, parameters for obtaining a higher accuracy in a specific range of ranges may be chosen (e.g. if an object between 4 or 5 meters distant from the radar should be detected, the radar parameters may be adapted to search for movement signature within that distance range).

In this embodiment, it may first be determined whether one or more persons walk inside a supervised environment. Based on this information, it may already be determined whether an anomalous situation is detected. For example if no walking movement is detected, a second radar scan may be performed, searching for a breathing movement, i.e. a higher frequency/smaller displacement range than that of the walking movement. Based on a result of the second scan, it may again be determined whether an anomalous situation is present. Based on an evaluation of the results of both scans, the processing unit may then generate the monitoring signal.

Additionally or alternatively, also multiple scans for performing walking movement detection and/or breathing movement detection may be performed respectively. For example, in case during a walking movement detection a movement is detected but cannot be clearly classified as a detected walking movement (e.g. the radar parameters are not exactly within thresholds for which a walking movement is classified), another scan for detecting walking movement may be performed, possibly with a different orientation or a different position of the monitoring device.

According to at least one advantageous embodiment, the method further comprises at least one of:
receiving, via a modem, a starting signal to perform a monitoring routine;
returning, by the autonomous robot unit, to a docking station of the monitoring device.

### BRIEF DESCRIPTION OF THE DRAWINGS

Further advantageous embodiments are described in the attached claims and the following description of the figures.

FIG. 1 shows a structural diagram of a monitoring device according to one embodiment;

FIG. 2 shows a structural diagram of a monitoring device according to another embodiment;

FIG. 3 shows a perspective view of a monitoring device according to one embodiment;

FIG. 4 shows a flowchart of a method for operating a monitoring device according to one embodiment; and

FIG. 5 shows a flowchart of a method for operating a monitoring device according to another embodiment.

### DETAILED DESCRIPTION OF ILLUSTRATIVE EMBODIMENTS

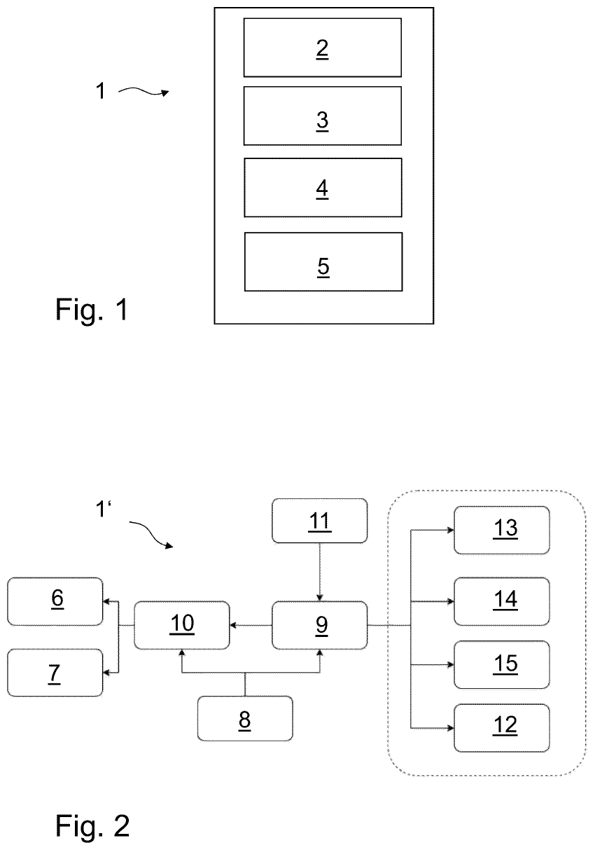

FIG. 1 shows a structural diagram of a monitoring device 1 according to one embodiment of the disclosure. The monitoring device 1 comprises an autonomous robot unit 2, a radar sensor unit 3, and a processing unit 4.

The autonomous robot unit 2 is arranged to navigate the monitoring device 1 along a navigation path. To do so, the autonomous robot unit 2 may comprise means for moving the monitoring device 1, such as for example a motor and wheels, and means for navigation, such as for example a positioning module. The navigation path may be pre-stored in a memory unit 5 of the monitoring device 1, or may be calculated by the autonomous robot unit 2 during navigation.

The radar sensor unit 3 is arranged to detect a person using radar technology. Using radar technology, the radar sensor unit 3 is able to detect a person for example behind a wall and/or behind a door and/or behind other objects. Therefore, the monitoring device 1 may be navigating along a navigation path in a corridor of a nursing home or a hospital, for example, and may detect persons inside rooms through walls and/or closed doors.

The radar sensor unit 3 may be in particular capable of inferring information regarding a position of a detected person within a room as well as a movement of the detected person. Collected information may comprise range, displacement range, frequency, azimuth angle, and elevation angle about the movement.

The at least one processing unit 4 is arranged to detect an anomalous situation based on a detection result of the radar sensor unit 3. For example, an anomalous situation may be detected in a situation in which a person should be detected but cannot be detected, i.e. it is assumed that the person is missing. Or that a person is detected where no person should be detected, or a number of persons detected is higher than a number of persons that should be detected.

The at least one processing unit 4 is further arranged to generate a monitoring signal in case an anomalous situation has been detected. The monitoring signal can be used to trigger an alarm, either on the monitoring device 1 itself, or by sending the monitoring signal to a monitoring system.

The autonomous robot unit 2 and the radar sensor unit 3 may use, for processing, either the processing unit 4 of the monitoring device 1 or may have individual processing means, such as a microprocessor, included or a combination thereof.

Algorithms and additional information for performing navigation of the monitoring device 1 and for performing the person detection using the radar sensor unit 3 may be stored in the memory unit 5.

With the above-described monitoring device 1, persons may be detected within rooms without entering those rooms, without opening doors, and even without installing parts of a monitoring system inside those rooms. This provides a very efficient way to perform safety checks on residents or patients in nursing rooms or hospitals, without unnecessarily invading their privacy.

FIG. 2 shows a structural diagram of a monitoring device 1′ according to a more detailed embodiment.

The monitoring device 1′ comprises a DC motor unit 6 with DC motors and encoders to drive differential drive wheels, not shown herein.

Moreover, the monitoring device 1′ comprises an infrared sensor unit 7, which may be used for docking the monitoring device 1′ to a station, for example for charging a battery of the monitoring device 1′ not shown herein.

The monitoring device further comprises a power management system unit 8, which is arranged to provide the entities of the monitoring system 1′ with power.

The monitoring device 1′ further comprises a single board computer, SBC, 9 and a microcontroller 10. Both may be part of a processing unit of the monitoring device 1′ and maybe arranged to perform all processing operations for navigating the monitoring device and for detecting persons, detecting anomalous situations and generating a monitoring signal. In particular, the microcontroller 10, a low power processor with real-time capabilities, is performing odometry control and controls the DC motor unit 6. The SBC 9 performs, for example, navigation algorithms, scanning algorithms, and other processing algorithms required for running the monitoring device 1′.

The monitoring device 1′ further comprises a modem 11, which is arranged to provide a network access for the monitoring device 1′ to a network. This modem is an interface via which the monitoring device 1′ may receive and send messages.

Via the modem 11, the monitoring device 1′ may be connected to a mobile application, which may be used by a user to control the monitoring device 1′, for example using a smartphone, a tablet, a computer, etc. Via the modem 11, a user may communicate with the monitoring device 1′ using the mobile application, for example to instruct the monitoring device 1′ to initiate a monitoring routine, to manage the behavior of the monitoring device 1′ and information, e.g. the monitoring signal, received from it.

The monitoring device 1′ further comprises an ultra-wideband, UWB, radar sensor 12, via which the monitoring device 1′ may perform UWB radar scans of an environment of the monitoring device 1′. With said UWB radar sensor 12, persons behind walls and closed doors may be detected. In particular, the UWB radar sensor 12 may be arranged to perform Doppler-range readings with dynamic thresholds, e.g. multiple versions of CFAR, to detect human movements. Radar imaging and analysis of multi-path reflections dependent on the environment in which the monitoring device 1′ is located may be used to provide a detection of a person with a high accuracy.

In particular, the UWB radar sensor 12 may be arranged to detect a range of a movement of a human being, a velocity of a movement of a human being, an angle at which a human being is detected with respect to a location of the monitoring device 1′, a distance from the monitoring device 1′ at which a human being is detected and information regarding a height above ground at which a movement of a human being is detected (therefore being able to locate a detected person in the scanned environment with high accuracy).

The UWB radar sensor 12 therefore is arranged to scan a room using UWB radar technology and thereby detecting a movement signature of a detected person. Therewith, movements as small as breathing can be detected and used to determine target position within the room.

The detected movement signature may then be analyzed using the processing unit of the monitoring device 1′, in particular using the single board computer 9. Thereby, the presence of quick movements of a high radar cross-section, RCS, i.e., the part of the human body that reflects the radar waves most directly, may be determined as a person walking.

In case no such walking movement is detected, the UWB radar sensor 12 may perform a second scan to search for breathing signatures, i.e. a small periodic movement from the chest or abdominal displacement. The human physiology provides constraints of frequencies and displacement size for the motion, which enable the classification of “breathing movement” or “walking movement” and allow relatively accurate positioning of a detected person. Depending on the distance and angular position of the detected person, it may be classified whether the detected person is within an area of the room defined as “bed area”, out of bed or lying on the floor, etc. Based on the above, it may be determined whether an anomalous situation is detected or not. An absence of detections may also be classified as anomalous, in case a person should be detected, since it could potentially represent an emergency situation i.e., person not in the room or not breathing.

The monitoring device 1′ further comprises a stereo camera system 13 including an RGB front camera, a 2D LIDAR sensor 14, and a mono RGB back camera 15. Those may be used by the monitoring device 1′ to perform navigation in the environment, in which the monitoring device 1′ is used. In particular, the mentioned cameras and sensors may be used to circumnavigate obstacles such as objects or persons on a corridor, in which the monitoring system 1′ navigates, and to find points of interest, i.e. positions at which a scan with the UWB radar sensor 12 may be performed (such as a door for example), or a docking station for the monitoring device 1′, etc.

The monitoring device 1′ as shown in FIG. 2 is a possible implementation of the monitoring device 1 according to FIG. 1. In this possible implementation according to FIG. 2, for example, the DC motor unit 6, the infrared sensor unit 7, the stereo camera system 13, the 2D-LIDAR sensor 14, and the mono back camera 15 may be considered being parts of the autonomous robot unit 2. The UWB radar sensor may be considered being part of the radar sensor unit 3, and the microcontroller 10 and the SBC 9 may be considered being part of the processing unit 4. Other implementations, however, are of course also possible.

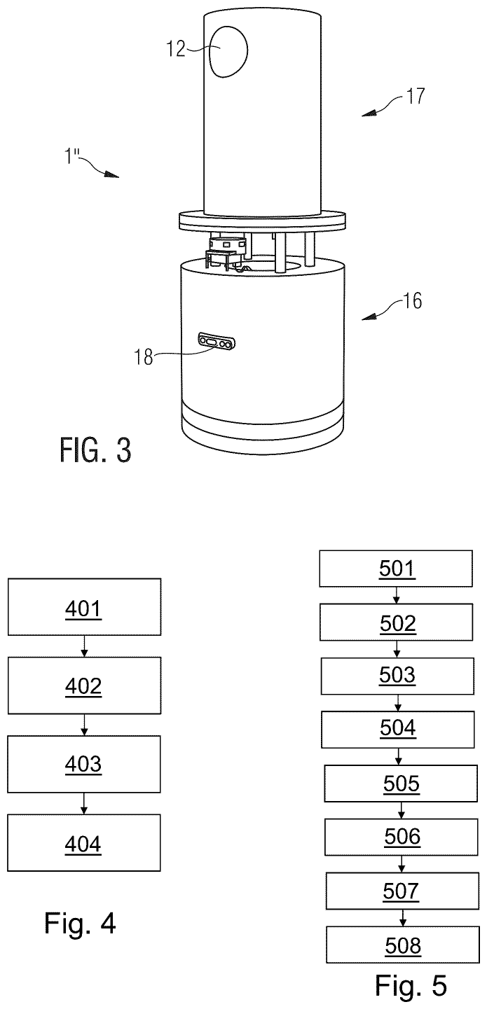

FIG. 3 shows a perspective view of a monitoring device 1″ according to an embodiment. The monitoring device 1″ comprises a lower part 16 and an upper part 17, each covering approximately half of an entire height of the monitoring device 1″.

The lower part 16 comprises a depth camera 18, a 2D-LIDAR sensor 14 and further components inside or below a casing not visible in FIG. 3, such as other components of a processing unit and of an autonomous robot unit described within this application, for example with reference to FIGS. 1 and 2.

The upper part 17 comprises an UWB radar sensor 12. The arrangement of the components of the autonomous robot unit being located at the lower part is advantageous since this way even small obstacles in the way of a path of the monitoring device 1″ can be detected and avoided. The UWB radar sensor being part of a radar sensor unit being arranged at the upper part 17 is advantageous since the height of the radar sensor, therefore, is located further away from a ground, reducing multipath reflections. The radar scans for detecting persons, therefore, achieve a high accuracy and reliability. A height of the monitoring device 1″ shown in FIG. 3 is, for example, about 90 cm. The UWB radar sensor 12 is, therefore, located approximately at a height above ground of 80 cm to 85 cm. Other sizes of such monitoring device are, of course, also possible.

FIG. 4 shows a flowchart of a method for operating a monitoring device according to one embodiment. The method may be used, for example, to operate the monitoring devices 1, 1′, 1″ according to FIG. 1, 2, or 3.

In a step 401, an autonomous robot unit of the monitoring device navigates the monitoring device along a navigation path. The navigation path may be pre-stored or may be determined using any of navigation algorithms, positioning modules, sensors, etc.

The navigation of the monitoring device may be performed using, for example, Simultaneous Localization and Mapping, SLAM, and known navigation algorithms. The autonomous robot unit may further be operated using the Robot Operating System, ROS.

In a step 402, a radar sensor unit of the monitoring device performs at least one radar scan to detect a person. The scan is performed using radar technology, for example UWB radar technology, or another radar technology. The scan may be performed while the monitoring device is at a stop, e.g. at a point of interest such as in front of a door, or may be performed while the monitoring device is navigating along the path.

Instead of only one scan, also multiple scans of a certain area—either from one position using a same orientation of the monitoring device, or from one position using different orientations of the monitoring device, or from different positions along the path—may be performed, in order to obtain more reliable results by increasing accuracy and resolution of the radar scans therewith.

In a step 403, using a processing unit of the monitoring device, it is determined whether an anomalous situation is detected based on a detection result of the radar sensor unit.

In a step 404, using the processing unit, a monitoring signal is generated in case an anomalous situation has been detected. Additionally, it may also be possible to generate another monitoring signal in case no anomalous situation has been detected, e.g. a signal to inform a supervisor of the monitoring device that a checked area, e.g. a scanned room, is in order and no anomalous situation was detected.

With this method described herein, a detection of a person, and in particular a detection of a movement of a person (e.g. using Doppler radar sensing) may be achieved, from which it may be determined whether a person may need assistance or not. Additionally, positioning information regarding a detected person may be considered for determining whether assistance may be needed.

Operating a monitoring device with such method as described herein, for example during night-time in a nursing home or hospital may provide significantly increased security for residents or patients, since checks with such monitoring device may be performed at a much higher frequency than working staff may be able to provide. Additionally, the monitoring proposed herewith is significantly less intrusive than existing monitoring systems, which rely on sensors and/or cameras installed in residents and patients'rooms.

FIG. 5 shows a flowchart of a method for operating a monitoring device according to another embodiment. The method may be used, for example, to operate the monitoring devices 1, 1′, 1″ according to FIG. 1, 2, or 3.

In a step 501, a caregiver of a nursing home or hospital activates the monitoring device, for example at night-time, via a smartphone application.

In a step 502, the monitoring device leaves its charging station and begins autonomously patrolling the corridor, moving from room-to-room and stopping outside closed doors.

In a step 504, the monitoring device uses a radar sensor unit to scan through the closed door and/or walls and locates a person within the room. The monitoring device may also reposition itself if alternative detection angles are required.

In a step 505, a search for movement signatures within the room is performed, from large movements (e.g. walking movements) to small movements (e.g. breathing movements). In a step 506, once a movement signature is identified, the monitoring device determines, using a processing unit, whether the location and position of the movement signature is anomalous for that particular room.

In a step 507, if a situation is determined to be anomalous, a notification is sent through an escalation path designed for caregivers. The caregivers receive a notification via their smartphone or other internet enabled devices and can choose whether to perform a physical check of that room.

In a step 508, the monitoring device continues to operate by repeating steps 502 (i.e., the navigation and stopping in front of rooms) to 507 throughout the night, until the caregivers initiate a docking and charging sequence from their smartphone, for example at an end of a shift.

In a step 509, the monitoring device returns to its charging station.

The advantages describe herein with regard to nursing homes and hospitals may, however, of course also be useful in other fields, which is why the monitoring device and method to operate such described in this application may also improve property security, e.g. to identify trespassers, etc.

1.-10. (canceled)

11. A monitoring device comprising:
an autonomous robot unit configured to navigate the monitoring device along a navigation path;
a radar sensor unit configured to detect a person using radar technology; and
at least one processing unit configured to:
detect an anomalous situation based on a detection result of the radar sensor unit, and
generate a monitoring signal in response to detecting the anomalous situation.

12. The monitoring device according to claim 11, wherein the radar sensor unit comprises at least one ultra-wideband (UWB) radio sensor.

13. The monitoring device according to claim 12, wherein the radar sensor unit is configured to determine at least one of the following:
a range at which a movement of a detected person is located,
a displacement range of a movement of a detected person,
a frequency of a movement of a detected person,
an azimuth angle at which a movement of detected person is located, or
an elevation angle at which a movement of a detected person is located.

14. The monitoring device according to claim 12, wherein the radar sensor unit is configured to detect, by Doppler radar detection, a walking movement of a person and/or a breathing movement of a person.

15. The monitoring device according to claim 11, wherein the autonomous robot unit comprises at least one of the following:
a light imaging, detection and ranging (LIDAR) sensor,
a depth sensor,
an RGB camera, or
a positioning sensor.

16. The monitoring device according to claim 11, wherein the radar sensor unit is configured to detect, by Doppler radar detection, a walking movement of a person and/or a breathing movement of a person.

17. The monitoring device according to claim 16, wherein the radar sensor unit is configured to determine at least one of the following:
a range at which a movement of a detected person is located,
a displacement range of a movement of a detected person,
a frequency of a movement of a detected person,
an azimuth angle at which a movement of detected person is located, or
an elevation angle at which a movement of a detected person is located.

18. The monitoring device according to claim 11, wherein the radar sensor unit is configured to determine at least one of the following:
a range at which a movement of a detected person is located,
a displacement range of a movement of a detected person,
a frequency of a movement of a detected person,
an azimuth angle at which a movement of detected person is located, or
an elevation angle at which a movement of a detected person is located.

19. The monitoring device according to claim 11, further comprising a modem configured to send the monitoring signal to a monitoring system.

20. The monitoring device according to claim 11,
wherein the autonomous robot unit is configured to navigate the monitoring device along the navigation path in an indoor environment, and
wherein the radar sensor unit is configured to detect a person behind a wall and/or a door.

21. A method for operating a monitoring device comprising an autonomous robot unit, a radar sensor unit and at least one processing unit, the method comprising:
navigating, by the autonomous robot unit, along a navigation path;
performing, by the radar sensor unit, at least one radar scan to detect a person using radar technology;
determining, by the processing unit, whether an anomalous situation is detected based on a detection result of the radar sensor unit; and
generating, by the processing unit, a monitoring signal in response to detecting the anomalous situation.

22. The method according to claim 21, wherein performing, by the radar sensor unit, the at least one radar scan to detect the person comprises:
performing, by the radar sensor unit, a first scan to detect a walking movement of a person; and
performing, by the radar sensor unit, a second scan to detect a breathing movement of the person.

23. The method according to claim 21, further comprising at least one of:
receiving, via a modem, a starting signal to perform a monitoring routine; or
returning, by the autonomous robot unit, to a docking station of the monitoring device.
